SEQUENCE LISTING (2 sequences)

SEQ ID NO: 1 - protein, 377 aa, Homo sapiens
MSPLNQSAEGLPQEASNRSLNATETSEAWDPRTLQALKISLAVVLSVITLATVLSNAFVLTTILLTRKLHTPANYLIGSLATTDLLVSILVMPISIAYTITHTWNFGQILCDIWLSSDITCCTASILHLCVIALDRYWAITDALEYSKRRTAGHAATMIAIVWAISICISIPPLFWRQAKAQEEMSDCLVNTSQISYTIYSTCGAFYIPSVLLIILYGRIYRAARNRILNPPSLYGKRFTTAHLITGSAGSSLCSLNSSLHEGHSHSAGSPLFFNHVKIKLADSALERKRISAARERKATKILGIILGAFIICWLPFFVVSLVLPICRDSCWIHPALFDFFTWLGYLNSLINPIIYTVFNEEFRQAFQKIVPFRKAS

SEQ ID NO: 2 - protein, 374 aa, Mus musculus
MSPPNQSLEGLPQEASNRSLNATGAWDPEVLQALRISLVVVLSVITLATVLSNAFVLTTILLTKKLHTPANYLIGSLATTDLLVSILVMPISIAYTTTRTWNFGQILCDIWVSSDITCCTASILHLCVIALDRYWAITDALEYSKRRTAGHAAAMIAAVWIISICISIPPLFWRQATAHEEMSDCLVNTSQISYTIYSTCGAFYIPSILLIILYGRIYVAARSRILNPPSLYGKRFTTAQLITGSAGSSLCSLNPSLHESHTHTVGSPLFFNQVKIKLADSILERKRISAARERKATKTLGIILGAFIICWLPFFVVSLVLPICRDSCWIHPALFDFFTWLGYLNSLINPVIYTVFNEDFRQAFQKVVHFRKIS

(19) United States
(12) Patent Application Publication
(10) Pub. No.: US 2026/0263449 A1
(43) Pub. Date: Sep. 10, 2026

(54) METHODS AND COMPOSITIONS FOR TREATING COCAINE ADDICTION

(71) Applicant: The Jackson Laboratory, Bar Harbor, ME (US)

(72) Inventors: Michael Saul, Bar Harbor, ME (US); Elissa Chesler, Bar Harbor, ME (US)

(73) Assignee: The Jackson Laboratory, Bar Harbor, ME (US)

(21) Appl. No.: 19/160,982

(22) PCT Filed: Feb. 29, 2024
(86) PCT No.: PCT/US2024/017816

§ 371 Date: Aug. 29, 2025

Related U.S. Application Data
(60) Provisional application No. 63/487,745, filed on Mar. 1, 2023.

(51) Int. Cl. A61K 31/496; A61K 47/12; A61K 47/14; A61K 47/22; A61K 47/44; A61P 25/30

(52) U.S. Cl. CPC A61K 31/496; A61K 47/12; A61K 47/14; A61K 47/22; A61K 47/44; A61P 25/30

(57) ABSTRACT

The disclosure relates to compositions and methods for treating cocaine addiction in a subject in need thereof utilizing a therapeutically effective amount of the hydrophobic 5-hydroxytryptamine receptor 1 D-specific agonist: (S)-3,4-dihydro-1-[2;[4-(4-methoxyphenyl)-1-piperazinyl]ethyl]-N-methyl-1 H-2-benzopyran-6-carboxamide, capable of crossing the blood-brain barrier. Further, the agonist alleviates volitional cocaine-taking behavior, facilitates recovery for cocaine addiction and prevents relapse into cocaine addiction.

### GOVERNMENT LICENSE RIGHTS

This invention was made with government support under P50 DA039841 and R01 DA037927 awarded by the National Institutes of Health. The government has certain rights in the invention.

### REFERENCE TO AN ELECTRONIC SEQUENCE LISTING

The contents of the electronic sequence listing (J022770138WO00-SEQ-HJD.xml; Size: 3,491 bytes; and Date of Creation: Feb. 29, 2024) is herein incorporated by reference in its entirety.

### BACKGROUND

Cocaine addiction is a chronic, relapsing disease characterized by a psychological and physiological inability to stop consuming cocaine, even though it causes psychological and/or physiological harm. Most treatments are psychosocial, designed to eliminate or decrease addictive behaviors and reduce the likelihood of relapse after successful treatment. The success rate of such treatments is low; therefore, addiction is also a costly personal and public health issue.

### SUMMARY

Some aspects of the present disclosure relate to a method of treating (e.g., alleviating one or more symptom of) addiction of commonly misused substances, such as cocaine, opioids, methamphetamine, alcohol, or nicotine. In some embodiments, the method comprises administering to the subject a composition comprising a hydrophobic 5-hydroxytryptamine receptor 1D (5-HTR1D)-specific agonist, wherein a therapeutically effective amount of the hydrophobic 5-HTR1D-specific agonist crosses the blood-brain barrier in the subject. Herein, a 5-HTR1D agonist is “specific” if it has a binding affinity for the 5-HTR1D receptor that is at least 1000-fold, at least 2000-fold, at least 3000-fold, at least 4000-fold, or at least 5000-fold greater than its binding affinity for the closely related 5-HTR1B receptor.

In some embodiments, the disclose relates to a method of treating cocaine addiction in a subject in need thereof, comprising administering to the subject a composition comprising a hydrophobic 5-HTR1D-specific agonist, wherein a therapeutically effective amount of the hydrophobic 5-HTR1D-specific agonist crosses the blood-brain barrier in the subject.

In some embodiments, the hydrophobic 5-HTR1D-specific agonist binds specifically to 5-HTR1D with a Ki of less than 1000 nM. In some embodiments, the hydrophobic 5-HTR1D-specific agonist binds specifically to 5-HTR1D with a Ki of less than 100 nM. In some embodiments, the hydrophobic 5-HTR1D-specific agonist binds specifically to 5-HTR1D with a Ki of less than 10 nM. In some embodiments, the hydrophobic 5-HTR1D-specific agonist binds specifically to 5-HTR1D with a Ki of less than 5 nM. In some embodiments, the hydrophobic 5-HTR1D-specific agonist binds specifically to 5-HTR1D with a Ki of less than 1 nM. In some embodiments, the hydrophobic 5-HTR1D-specific agonist binds specifically to 5-HTR1D with a Ki of about 0.5 nM to about 1 nM. In some embodiments, the hydrophobic 5-HTR1D-specific agonist binds specifically to 5-HTR1D with a Ki of about 0.8 nM to about 1 nM. In some embodiments, the hydrophobic 5-HTR1D-specific agonist binds specifically to 5-HTR1D with a Ki of about 0.9 nM. In some embodiments, the hydrophobic 5-HTR1D-specific agonist binds specifically to 5-HTR1D with a Ki of 0.9 nM.

In some embodiments, the hydrophobic 5-HTR1D-specific agonist comprises (or is) Compound I:

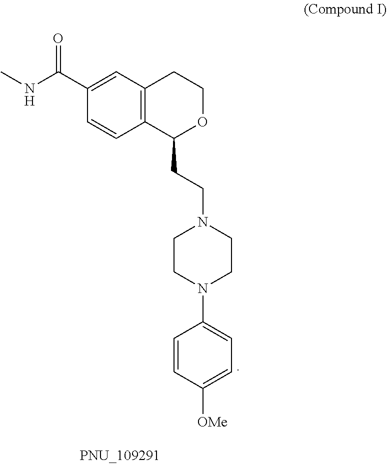 [SMILES: CNC(=O)c1ccc2c(c1)CCO[C@H]2CCN1CCN(c2ccc(OC)cc2)CC1]

In some embodiments, the composition does not comprise PNU-142633 ((1S)-1-[2-[4-[4-(aminocarbonyl)phenyl]-1-piperazinyl]ethyl]-3,4-dihydro-N-methyl-1H-2-benzopyran-6-carboxamide), which is a hydrophilic compound that is unable to cross the BBB.

In some embodiments, the therapeutically effective amount is about 1 mg/kg to about 100 mg/kg.

In some embodiments, the therapeutically effective amount is formulated for systemic administration or oral administration (e.g., the composition comprises an excipient typically used for systemic or oral administration).

In some embodiments, the composition further comprises a pharmaceutically acceptable excipient. For example, the excipient may be a polyethoxylated castor oil, such as Kolliphor®, for example, Kolliphor® HS-15. In some embodiments, the composition further comprises N-methyl-2-pyrrolidone (NMP) and/or citric acid.

In some embodiments, the excipient is equimolar to the hydrophobic 5-HTR1D-specific agonist.

In some embodiments, the subject is a human.

In some embodiments, the subject is a non-human preclinical animal model, such as a rodent, for example, a mouse.

In some embodiments, the subject is genetically predisposed to cocaine addiction and/or has been treated for cocaine addiction.

In some embodiments, the therapeutically effective amount is effective to alleviate volitional cocaine-taking behavior. In some embodiments, the therapeutically effective amount is effective to facilitate recovery for cocaine addiction. In some embodiments, the therapeutically effective amount is effective to prevent relapse into cocaine addiction.

Other aspects of the present disclosure relate to a composition comprising a hydrophobic 5-HTR1D-specific agonist and a polyethoxylated castor oil. In some embodiments, the polyethoxylated castor oil is Kolliphor®, for example, Kolliphor® HS-15. In some embodiments, the composition further comprises N-methyl-2-pyrrolidone (NMP). In some embodiments, the composition further comprises citric acid. In some embodiments, the composition excludes dimethyl sulfoxide (DMSO). In some embodiments, the excipient is equimolar to the hydrophobic 5-HTR1D-specific agonist.

Yet other aspects of the present disclosure relate to a method of treating cocaine addiction in a subject in need thereof, comprising administering to the subject a composition comprising a hydrophobic 5-HTR1D-specific agonist, wherein a therapeutically effective amount of the 5-HTR1D-specific agonist crosses the blood-brain barrier in the subject, and the 5-HTR1D-specific agonist binds specifically to 5-HTR1D with a Ki of less than 5 nM. In some embodiments, the 5-HTR1D-specific agonist binds specifically to 5-HTR1D with a Ki of less than 1 nM. In some embodiments, the 5-HTR1D-specific agonist binds specifically to 5-HTR1D with a Ki of about 0.8 nM to about 1 nM. In some embodiments, the 5-HTR1D-specific agonist binds specifically to 5-HTR1D with a Ki of about 0.9 nM. In some embodiments, the 5-HTR1D-specific agonist does not bind to 5-HTR1B. In some embodiments, the 5-HTR1D-specific agonist binds to 5-HTR1B with a Ki of greater than 5000 nM. In some embodiments, the 5-HTR1D-specific agonist binds to 5-HTR1B with a Ki of greater than 5000 nM and less than 10,000 nM.

### BRIEF DESCRIPTION OF THE DRAWINGS

FIG. 1 shows the internal temperature of mice before, during, and after intravenous injection with vehicle or a range of doses of the hydrophobic 5-HTR1D agonist PNU-109291. PNU-109291 induced central hypothermia at 10 mg/kg body weight, indicating that it penetrates the blood-brain barrier and acts as 5-HTR1D agonist at these doses. (Plotted: mean±standard error)

FIG. 2A shows the amount of cocaine taken by mice pre-treated with vehicle or a range of doses of PNU-109291. Intravenous pre-treatment with PNU-109291 reduced volitional cocaine-taking behavior at a dose of 10 mg/kg body weight. FIG. 2B shows the amount of inactive lever presses performed by mice pre-treated with vehicle or a range of doses of PNU-109291. There was no effect of intravenous PNU-109291 pre-treatment on inactive lever pressing. (Plotted: mean±standard error)

### DETAILED DESCRIPTION

The present disclosure, in some aspects, relates to the use of blood-brain barrier permeable agonists specific for 5-HT1D (such as the compound PNU-109291) to alleviate addiction-related behaviors. There is no FDA-approved pharmacotherapy for cocaine use disorder, for example. Additionally, presently used pharmacotherapies for opioid use disorder concentrate on replacing the drug with a similar drug that has different kinetics. The present disclosure, in some aspects, proposes an alternative therapy that targets the motivation to take an addictive drug, such as cocaine and/or opioids.

### Cocaine Addiction

There are currently over 21 million Americans who have a substance use disorder, and drug and alcohol addictions cost the US economy over $600 billion annually. Drug overdose deaths accounted for 70,237 deaths in the US during 2017 and cost the US economy over $193 billion annually (CDC, 2018). Cocaine use occurs among all demographic and socioeconomic groups and has been estimated to be second only to heroin according to ranking scores that have used evidence-based data to assess drug “harmfulness” (Nutt et al., (2007) The Lancet 369(9566):1047-1053). Furthermore, cocaine has been identified as one of the most powerful drug reinforcers known (Kuher et al. (1991) Trends in Neurosciences 14(7):299-302). Accordingly, cocaine addiction is costly; both in terms of mortality and economic impact.

Several theories have been put forward to explain the phenomenon of addiction, including unchecked impulsivity, reward deficiency, and maladaptive learning (i.e., the growing incentive salience of a drug's predictive cues with chronic use). Each of these theories is characterized by widespread changes in distinct circuit functions across the brain that are involved in a number of behavioral processes, such the encoding of rewarding stimuli or dopaminergic response pathways (Volkow et al. (2013) Curr Opin Neurobiol. 23(4):639-648). Accordingly, an important distinction in understanding addiction is the difference between volitional (i.e., impulse control)- and hedonic homeostasis (i.e., pleasure)-driven behaviors. Importantly, the brain circuits that mediate pleasurable effects of addictive drugs are anatomically, neurophysiologically, and neurochemically different from those mediating 140 physical dependence, and from those mediating craving and relapse (Blum et al. (2012) Curr Pharm Des. 18(1):113-118). Experimental tests to assess these different circuit-related behaviors in response to addiction have been developed in mice. For example, cocaine-addicted mice may be given the option to self-administer the drug by pressing a lever. By measuring whether these mice press the lever only if cocaine is subsequently delivered, it can be determined that the mice volitionally choose to press the lever under specific circumstances (i.e., when they have learned they will receive cocaine). Alternatively, if the mice press the lever even during time periods in which they do not receive cocaine, the addiction is likely to have altered brain circuitry associated with dysregulation of hedonic tone. In some embodiments, a subject treated in accordance with the present disclosure exhibits volitional control-associated addiction behaviors. In some embodiments, a subject treated in accordance with the present disclosure exhibits multiple addiction behaviors. Addiction behaviors may include, but are not limited to: mood swings, changed activity levels, altered motivation, decreased mental performance, social isolation, inability to meet obligations, and/or changes in personal relationships.

Genetic and environmental factors contribute to an individual's propensity for addiction; however, as the disease progresses, atypical neuronal plasticity may occur within certain brain circuits, leading to the development and maintenance of addiction. For example, the dopaminergic and glutamatergic circuits both have roles in the neuroplasticity associated with addiction; however, there are serotonergic mechanisms involved as well. It is thought that the reward circuitry of the brain may contribute to various addictive disorders and related disorders of reward behavior such as anorexia. Specifically, stimulation of the ventral tegmental area (VTA), a heterogeneous area of the brain predominantly composed of dopamine and 7-amino butyric acid (GABA) neurons, results in a release of dopamine to projection sites, such as the nucleus accumbens (NAc) (Cooper et al., (2017) Neurotherapeutics 14(3):687-697). In the NAc, the dopamine activates its receptors on medium spiny neurons (MSNs), which are GABAergic projection neurons consisting of two classes (D1- or D2-like dopamine receptors). D1 MSNs increase thalamocortical drive (e.g., rewarding stimuli), while D2 MSNs decrease thalamocortical drive (e.g., aversive stimuli). Optogenetically stimulating D2 MSNs reduces motivation for cocaine, highlighting the role of MSNs in volitional drug consumption (Bock et al. (2013) Nature Neuroscience 16(5):632-638). While activation of VTA dopamine has been found to be a major component of initial reward-related behaviors (e.g., drug-seeking behaviors), it is the NAc that seems to be heavily involved in the progression to addiction. Cocaine has been shown to elevate dopamine levels in synapses, activating dopamine receptors. In addition, cocaine affects serotonin receptors: for example, self-administered cocaine has been found to inhibit monoamine uptake, resulting in elevated extracellular monoamines, such as serotonin, in a number of structures, including the NAc, VTA, dorsal raphe nucleus, hippocampus, striatum, and cortex (Kirby et al., (2012) Neuropharmacology 61(3):421-432). This elevation, resulting from “acute” cocaine exposure, becomes less noticeable during chronic exposure, resulting in an increased drive for more cocaine. Withdrawal from cocaine results in decreased levels of serotonin throughout the brain, specifically in the NAc, leading to the symptoms described below.

As addictive disorders have a strong neurological component, discontinuing use of an addictive substance or an addictive behavior generally results in physiological and/or psychological withdrawal symptoms. Therefore, in some embodiments of the present disclosure, a subject is experiencing withdrawal symptoms. In some embodiments, a subject is experiencing physiological, psychological, or a combination of physiological and psychological symptoms of withdrawal. Physiological symptoms of withdrawal include, but are not limited to, grand mal seizure, heart attack, stroke, hallucination, delirium tremens, headaches, dizziness, chest tightness, difficulty breathing, arrhythmia, palpitation, nausea, vomiting, diarrhea, stomachache, muscle tension, twitches, tremors, shakes, muscle aches, sweating, and tingling sensations. Psychological symptoms of withdrawal include, but are not limited to, anxiety (e.g., generalized anxiety, panic attack, restlessness, irritability), depression (e.g., social isolation, fatigue, poor appetite), sleep abnormalities (e.g., insomnia, difficulty falling asleep or staying asleep), and cognitive difficulties (e.g., poor concentration, poor memory).

There are a number of different addictions, generally classified either as substance use addictions or behavior addictions (e.g., food, gambling, video gaming, sex, shopping, etc.). In some embodiments, a subject treated in accordance with the present disclosure has a substance use addition. Substance use addictions are characterized by a loss of control over substance use, compulsive substance seeking and craving, and continued use despite negative consequences. A person addicted to a substance typically also experiences physiological and/or psychological dependence on the substance.

In some embodiments, a subject is addicted to cocaine, a psychostimulant. Psychostimulants are a broad class of sympathomimetic drugs that cause euphoria, increased alertness, and increased physical capacity. Most are non-prescription drugs; however, some, such as ADDERALL® (amphetamine and dextroamphetamine), are prescription drugs. Other examples of psychostimulants include: amphetamines (e.g., methamphetamine, ephedrine, cathinone, phentermine, mephentermine, bupropion, methoxyphenamine, selegiline, amfepramone, pyrovalerone, MDMA (ecstasy), DOM (STP)), atomoxetine, dextroamphetamine, methylphenidate, modafinil, lisdexamfetamine, oxymetazoline, pseudoephedrine, phenylephrine, and anabolic steroids. Psychostimulants increase dopamine transmission; however, chronic use results in a reduction of dopamine activity, leading to dysregulation of the brain reward system and dysphoria. Some pharmacotherapeutics, such as desipramine, amantadine and bromocriptine, have been shown to decrease cocaine withdrawal symptoms. While the psychostimulant class of drugs broadly cause euphoria, the mechanisms by which this occurs differ between drugs. For example, although cocaine and amphetamines both act on presynaptic monoamine reuptake transporters, cocaine acts as a reuptake inhibitor (i.e., it blocks the action of the reuptake transporter, thus allowing more neurotransmitter to stay active in the synapse), whereas amphetamines are releasers (i.e., they are taken up by the transporter in exchange for neurotransmitter release into the synapse) (Ciccarone et al. (2011) Primary Care 38(1):41-58).

Although counseling remains the treatment of choice for cocaine dependence, many cocaine-dependent patients do not respond completely to standard drug counseling, and show only modest results. In addition, there are no registered pharmacological treatments to date, despite the wide range of medications tested for this type of dependence. Therefore, the development of new and more effective drug treatments for cocaine dependence is a research priority.

### Modulation of the Serotonin 1D Receptor

In some aspects, the present disclosure provides methods of treating or preventing cocaine addiction in a subject by administering an agent that modulates serotonin 1D receptor (5-HTR1D) activity. The natural ligand for 5-HTR1D is serotonin (5-hydroxytryptamine, i.e., 5-HT), which is released in response to cocaine administration. Htr1d, a gene encoding the serotonin 1D receptor, was found to be responsive to cocaine exposure in a subset of mice of diverse genetic backgrounds (International Patent Application No. PCT/US2020/65941). 5-HTR1D acts on 5-HTR1D within the central nervous system. Serotonin 1D receptors are primarily located in the basal ganglia, hippocampus, cortex, spinal cord, and vascular smooth muscle cells. Ligand binding causes a conformational change that triggers signaling via guanine nucleotide-binding proteins (G proteins), modulating the activity of downstream effectors, such as adenylate cyclase. Specifically, signaling through the receptor inhibits adenylate cyclase activity. The receptor also regulates the release of serotonin in the brain, affecting neural activity, and may also regulate the release of other neurotransmitters. The receptor is thought to play a role in neuropsychiatric disorders, such as depression, and induces vascular vasoconstriction in the brain. Without wishing to be bound by theory, it is thought that modulating (e.g., increasing or decreasing) the activity and/or expression of the serotonin 1D receptor may treat or prevent addiction, withdrawal, and/or relapse.

In some embodiments, the HTR1D protein is a Homo sapiens HTR1D, encoded by HTR1D (e.g., Gene ID: 3352). HTR1D is also known as G protein-coupled, serotonin 1D alpha receptor, serotonin receptor 1D, 5-HT-1D-alpha, 5-HT-1D, HTR1DA, HTRL, HTR1DA, and RDC4. The sequence of human HTR1D and murine HTRD1 are provided below:

HTR1D [Homo sapiens] (NP_000855.1)
(SEQ ID NO: 1)

| | |
|---|---|
| 1 | MSPLNQSAEG LPQEASNRSL NATETSEAWD PRTLQALKIS |
| | LAVVLSVITL ATVLSNAFVL |
| 61 | TTILLTRKLH TPANYLIGSL ATTDLLVSIL VMPISIAYTI |
| | THTWNFGQIL CDIWLSSDIT |
| 121 | CCTASILHLC VIALDRYWAI TDALEYSKRR TAGHAATMIA |
| | IVWAISICIS IPPLFWRQAK |
| 181 | AQEEMSDCLV NISQISYTIY STCGAFYIPS VLLIILYGRI |
| | YRAARNRILN PPSLYGKRFT |
| 241 | TAHLITGSAG SSLCSLNSSL HEGHSHSAGS PLFFNHVKIK |
| | LADSALERKR ISAARERKAT |
| 301 | KILGIILGAF IICWLPFFVV SLVLPICRDS CWIHPALFDF |
| | FTWLGYLNSL INPIIYTVEN |
| 361 | EEFRQAFQKI VPERKAS |

HTRID [Mus musculus] (AAI03537.1)
(SEQ ID NO: 2)

| | |
|---|---|
| 1 | MSPPNQSLEG LPQEASNRSL NATGAWDPEV LQALRISLVV |
| | VLSVITLATV LSNAFVLTTI |
| 61 | LLTKKLHTPA NYLIGSLATT DLLVSILVMP ISIAYTTTRT |
| | WNFGQILCDI WVSSDITCCT |
| 121 | ASILHLCVIA LDRYWAITDA LEYSKRRTAG HAAAMIAAVW |
| | IISICISIPP LFWRQATAHE |
| 181 | EMSDCLVNTS QISYTIYSTC GAFYIPSILL IILYGRIYVA |
| | ARSRILNPPS LYGKRFTTAQ |
| 241 | LITGSAGSSL CSLNPSLHES HTHTVGSPLF FNQVKIKLAD |
| | SILERKRISA ARERKATKTL |
| 301 | GIILGAFIIC WLPFFVVSLV LPICRDSCWI HPALFDFFTW |
| | LGYLNSLINP VIYTVENEDE |
| 361 | RQAFQKVVHF RKIS |

In some embodiments, the agent is an agonist. Agonist agents are those that partially or fully promote, induce, increase, and/or activate a biological activity of a native polypeptide disclosed herein (e.g., 5-HTR1D). In some embodiments, the agent simulates the natural activity of a native polypeptide (e.g., 5-HTR1D). In some embodiments, the agent is an agonist antibody.

In some embodiments, the agent is a hydrophobic 5-HTR1D-selective agonist, that is, the agent binds specifically to 5-HTR1D with a Ki of less than 1000 nM. In some embodiments, the hydrophobic 5-HTR1D-specific agonist binds specifically to 5-HTR1D with a Ki of less than 100 nM. In some embodiments, the hydrophobic 5-HTR1D-specific agonist binds specifically to 5-HTR1D with a Ki of less than 10 nm. In some embodiments, the hydrophobic 5-HTR1D-specific agonist binds specifically to 5-HTR1D with a Ki of less than 1 nM. In some embodiments, the hydrophobic 5-HTR1D-specific agonist binds specifically to 5-HTR1D with a Ki of about 0.5 nM to about 1 nM. In some embodiments, the hydrophobic 5-HTR1D-specific agonist binds specifically to 5-HTR1D with a Ki of about 0.8 nM to about 1 nM. In some embodiments, the hydrophobic 5-HTR1D-specific agonist binds specifically to 5-HTR1D with a Ki of about 0.9 nM. In some embodiments, the hydrophobic 5-HTR1D-specific agonist binds specifically to 5-HTR1D with a Ki of 0.9 nM.

In some embodiments, the hydrophobic 5-HTR1D-specific agonist has a binding affinity for the 5-HTR1D receptor that is at least 1000-fold, at least 2000-fold, at least 3000-fold, at least 4000-fold, or at least 5000-fold greater than its binding affinity for the closely related 5-HTR1B receptor.

In some embodiments, the hydrophobic 5-HTR1D-specific agonist binds to 5-HTR1B with a Ki of greater than 5000 nM. In some embodiments, the hydrophobic 5-HTR1D-specific agonist binds to 5-HTR1B with a Ki of about 5000 nM to about 10000 nM.

In some embodiments, the hydrophobic 5-HTR1D-selective agonist PNU-109,291 ((S)-3,4-Dihydro-1-[2-[4-(4-methoxyphenyl)-1-piperazinyl]ethyl]-N-methyl-1H-2-benzopyran-6-carboxamide), described elsewhere herein.

### Blood-Brain Barrier-Permeable Therapies

As used herein, the term “blood-brain barrier” (BBB) is used in reference to the unique properties of the microvasculature of the central nervous system (CNS). CNS vessels are continuous, non-fenestrated vessels that contain a series of additional properties which allow them to tightly regulate the movement of molecules, ions, and cells between the blood and the CNS. This selectivity arises from the epithelial-like tight junctions within the brain capillary endothelium. The BBB is anatomically and functionally distinct from the blood-cerebrospinal fluid barrier at the choroid plexus. This heavily restricting barrier capacity allows BBB endothelial cells to tightly regulate CNS homeostasis, which is critical to allow for proper neuronal function, as well as protect the CNS from toxins, pathogens, inflammation, injury, and disease. BBB dysfunction can lead to ion dysregulation, altered signaling homeostasis, as well as the entry of immune cells and molecules into the CNS, processes that lead to neuronal dysfunction and degeneration. The restrictive nature of the BBB, while imperative for proper physiological functions, provides an obstacle for drug delivery to the CNS, and, thus, major efforts have been made to generate methods to modulate or bypass the BBB for delivery of therapeutics.

Certain small molecule drugs may cross the BBB, for example, via lipid-mediated free diffusion, providing the drug is hydrophobic, has a molecular weight<400 Da, and forms<8 hydrogen bonds, chemical properties that are lacking in the majority of small molecule drugs and all large molecule drugs. Nevertheless, drugs can be engineered for BBB transport, based on the knowledge of the endogenous transport systems within the BBB. Because the BBB allows the diffusion of hydrophobic molecules while restricting hydrophilic molecules, engineering drugs to cross the BBB can include increasing the lipid solubility of the drug.

There are several ways to test whether a compound crosses the BBB. For example, microdialysis, autoradiography, and even whole-brain homogenization can be used in animal models. Because these methods are not feasible in human subjects, clinical trials use other techniques such as positron emission tomography, single-photon emission computerized tomography, and cerebrospinal fluid (CSF) sampling to indirectly measure BBB penetration. However, these methods are unnecessarily invasive and/or expensive. Therefore, a non-invasive, inexpensive, and simple method to determine BBB penetration was developed based on the finding that injection of a specific 5-HTR1D agonist GR46611 directly into the lateral ventricles of guinea pigs caused central hypothermia, but sumatriptan (a nonselective 5-HTR1D agonist not thought to be BBB permeable) injected subcutaneously does not (Skingle, Higgins, and Feniuk 1994). These results indicated the ability to measure 5-HTR1D agonists acting in the CNS by measuring hypothermia. Relative to cocaine intravenous self-administration, which requires an expensive surgery to implant a jugular catheter, substantial recovery time, and a labor-intensive multi-week behavioral testing paradigm, this hypothermia bioassay is inexpensive and takes little time. In some embodiments, the hypothermia bioassay described herein is used to determine whether a drug passes the BBB.

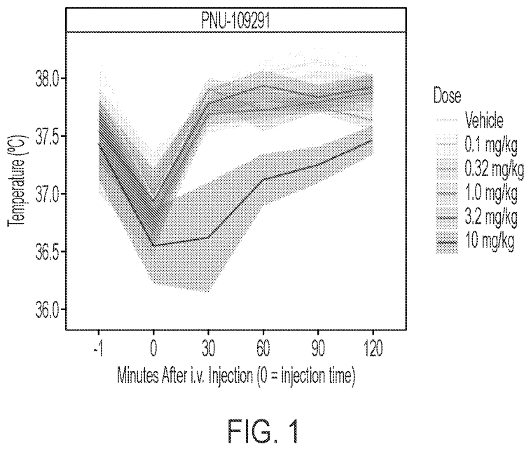

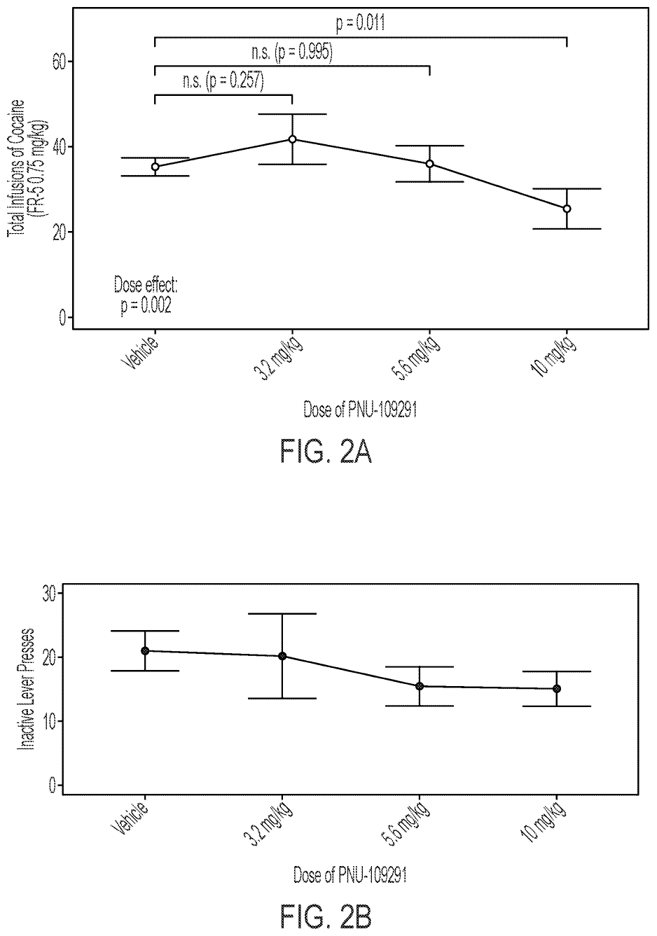

PNU-109291 (Compound I) is a potent and highly selective 5-HTR1D agonist that displays >600-fold selectivity over 5-HT1A and 5-HT2A receptors and no activity at 5-HT1B, 5-HT1E, 5-HT2B, 5-HT2C, 5-HT6 and 5-HT7 receptors, thus making it an attractive candidate drug for treatment of cocaine addiction through selective 5-HTR1D agonism. Furthermore, PNU-109291 is hydrophobic, an important property of BBB-permeable molecules. Surprisingly, the intravenous administration of PNU-109291 in mice caused central hypothermia at levels comparable to a known BBB-permeable drug (FIG. 1), indicating the ability of PNU-109291 to cross the BBB and act on the cells of the CNS. Moreover, to test its effect as a treatment of cocaine addiction, PNU-109291 was administered to cocaine-addicted mice. Remarkably, treatment with PNU-109291 led to a dose-dependent decrease in cocaine self-administration (FIG. 2A). PNU-109291 thus shows clear promise as a potential blood-brain barrier permeable pharmacotherapy for volitional aspects of addiction. In some aspects of the methods described herein, PNU-109291 is the BBB-permeable 5-HTR1D-specific agonist used to treat cocaine addiction.

### Methods of Preventing/Treating Addiction

Provided herein, in some embodiments, are methods of preventing or treating cocaine addiction disorder in a subject (e.g., a human subject), the method comprising, for example, administering to the subject a composition comprising a hydrophobic 5-HTR1D-specific agonist, wherein a therapeutically effective amount of the 5-HTR1D-specific agonist crosses the blood-brain barrier in the subject.

A subject herein may be a mammalian subject, such as a human subject. In some embodiments, a subject is a non-human primate or a rodent (e.g., mouse or rat), for example, used as animal models. In some embodiments, the subject has an addictive disorder (e.g., a substance use disorder). In some embodiments, the subject is genetically predisposed to addiction. Individuals who are predisposed to addiction include those having Reward Deficiency Syndrome (RDS), which encompasses dopamine resistance and therefore sensory deprivation of the reward circuitry (e.g., pleasure). In some embodiments, the subject has been treated for an addiction (i.e., the subject has an addictive disorder previously). In some embodiments, the subject has one addiction, i.e., the subject is addicted to one substance/behavior or class of substances (e.g., psychostimulants). In some embodiments, the subject is addicted to more than one (e.g., 2, 3, 4, or more) substances or classes of substances. In some embodiments, the subject has at least one substance use disorder and at least one behavioral addiction.

Non-limiting examples of routes of administration include oral (e.g., tablet, capsule, or liquid), intravenous, subcutaneous, inhalation, intranasal, intrathecal, intramuscular, intraarterial, and intraneural. In some embodiments, an agent that modulates serotonin 1D receptor expression and/or activity (e.g., PNU-109291) is administered as a tablet. In other embodiments, an agent that modulates serotonin 1D receptor expression and/or activity (e.g., PNU-109291) is administered subcutaneously.

In some embodiments, the agent that modulates HTR1D activity (e.g., PNU-109291) may be administered as part of a composition with a pharmaceutically acceptable excipient. A pharmaceutically acceptable excipient is a pharmacologically inactive material used together with a pharmacologically active material (e.g., HTR1D modulatory agent) to formulate a pharmaceutical composition. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Any one of the compositions provided herein may include a pharmaceutically acceptable excipient or carrier. In some embodiments, the pharmaceutically acceptable carrier is hydrophobic and facilitates BBB transport. In some embodiments, the pharmaceutically acceptable carrier causes central hypothermia when administered alone (e.g., dimethyl sulfoxide (DMSO)). In some embodiments, the pharmaceutically acceptable carrier does not cause central hypothermia when administered alone. In some embodiments, a pharmaceutically acceptable excipient comprises a pH-independent non-ionic surfactant. In some embodiments, a pharmaceutically acceptable excipient comprises 12-hydroxystearic acid (lipophilic moiety) and polyethylene glycol (hydrophilic moiety). In some embodiments, a pharmaceutically acceptable excipient comprises Kolliphor®. In some embodiments, a pharmaceutically acceptable excipient comprises Kolliphor® HS-15 (CAS Number 70142-34-6):

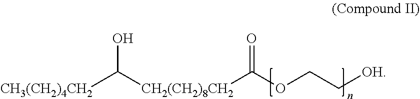

In some embodiments, a pharmaceutically acceptable excipient comprises N-methyl-2-pyrrolidone (NMP). In some embodiments, a pharmaceutically acceptable excipient comprises citric acid, for example, in saline. In some embodiments, a pharmaceutically acceptable excipient comprises 5% Kolliphor HS-15, 5% NMP, and citric acid in saline.

In some embodiments, a therapeutically effective amount of an agent may be administered to a subject to treat or prevent an addictive disorder, withdrawal, and/or relapse. The term treat, as known in the art, refers to the process of alleviating at least one symptom associated with a disease (e.g., addiction). A symptom may be a physiological, psychological, or pathological manifestation of the disease. Symptoms associated with addiction and/or withdrawal depend on the specific addiction, are described elsewhere herein. Treatment may also result in the prevention of a condition as provided herein, and therefore, includes prophylactic treatment (e.g., prevention of an addiction and/or prevention of a relapse). When used prophylactically, the subject is one in which a clinician expects that there is a likelihood for the development of an addiction (e.g., the subject is genetically predisposed to addiction and/or has previously been treated for an addiction).

To treat or prevent an addictive disorder, an agent that modulates the activity of the serotonin 1D receptor (e.g., PNU-109291) as provided herein should be administered as a therapeutically effective amount. As used herein, “therapeutically effective amount” refers to an amount of the composition or dose that produces one or more desired responses in the subject (e.g., reduces a subject's craving for an addictive substance, reduces symptoms of withdrawal, etc.). For in vivo purposes, the amount can be one that a clinician would believe may have a clinical benefit for a subject in need thereof. Methods are known for determining a therapeutically amounts of various therapeutic molecules (e.g., modulators of HTR1D activity). Amounts effective will depend on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the skilled medical practitioner. These factors are known to those of ordinary skill in the art and can be addressed with no more than routine experimentation.

As an example, in some embodiments, the agent is PNU-109291, and the dose is a 1, 3, 10, 30, 50, 75 or 100 mg tablet. In some embodiments, the dose is 2 mg, 4 mg, 5 mg, 15 430 mg, 20 mg, 25 mg, 35 mg, 40 mg, 55 mg, 60 mg, 65 mg, 70 mg, 80 mg, 85 mg, 90 mg, 95 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, or more (e.g., 5 mg-250 mg). In some embodiments, the dose is administered as a subcutaneous injection. Exemplary subcutaneous injection doses include 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, or more (e.g., 1 mg/mL-15 mg/mL).

An agent may be administered to a subject as a single dose or as multiple doses over the course of days, weeks, months, or years. The dose/dosage of an agent may be determined by a skilled medical practitioner, taking into consideration one or more factors, such as type and severity of disease as well as subject age, weight, height, sex, and ethnicity.

Any one of the methods of treatment provided herein may also include administration of an additional therapeutic, such as an additional addiction-specific treatment disclosed herein. Additional therapeutics for addiction or withdrawal (or symptoms thereof) may be administered to any one of the subjects provided herein. Any one of the methods provided herein may include the administration of one or more of these additional therapeutics. In some embodiments, the methods of treatment provided herein also include cognitive behavioral therapy and/or counseling in conjunction with any of the methods described herein.

### ADDITIONAL EMBODIMENTS

The present disclosure provides additional embodiments encompassed by the numbered paragraphs below:

Paragraph 1. A method of treating cocaine addiction in a subject in need thereof, comprising administering to the subject a composition comprising a hydrophobic 5-hydroxytryptamine receptor 1D (5-HTR1D)-specific agonist, wherein a therapeutically effective amount of the 5-HTR1D-specific agonist crosses the blood-brain barrier in the subject.

Paragraph 2. The method of Paragraph 1, wherein the 5-HTR1D-specific agonist binds specifically to 5-HTR1D with a Ki of less than 10 nM, less than 5 nM, or less than 1 nM, optionally less than about 0.9 nM.

Paragraph 3. The method of Paragraph 2, wherein the 5-HTR1D-specific agonist comprises Compound I:

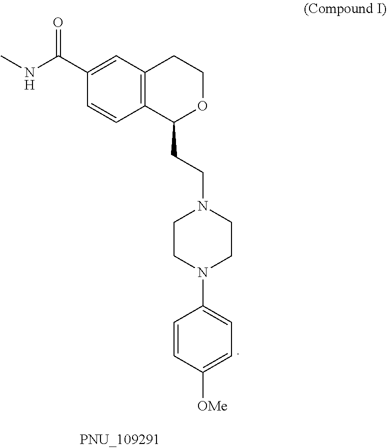 [SMILES: CNC(=O)c1ccc2c(c1)CCO[C@H]2CCN1CCN(c2ccc(OC)cc2)CC1]

Paragraph 4. The method of any one of the preceding Paragraphs, wherein the therapeutically effective amount is about 1 mg/kg to about 100 mg/kg.

Paragraph 5. The method of any one of the preceding Paragraphs, wherein the therapeutically effective amount is formulated for systemic administration or oral administration.

Paragraph 6. The method of any one of the preceding Paragraphs, wherein the composition further comprises a pharmaceutically acceptable excipient.

Paragraph 7. The method of Paragraph 6, wherein the excipient is a polyethoxylated castor oil, optionally Kolliphor®, preferably Kolliphor® HS-15.

Paragraph 8. The method of Paragraph 7, wherein the composition further comprises N-methyl-2-pyrrolidone (NMP).

Paragraph 9. The method of Paragraph 7 or 8, wherein the composition further comprises citric acid.

Paragraph 10. The method of any one of the preceding Paragraphs, wherein the excipient is equimolar to the 5-HTR1D-specific agonist.

Paragraph 11. The method of any one of the preceding Paragraphs, wherein the subject is a human.

Paragraph 12. The method of any one of Paragraphs 1-10, wherein the subject is a non-human preclinical animal model, optionally a rodent, preferably a mouse.

Paragraph 13. The method of any one of Paragraphs 1-11, wherein the subject is genetically predisposed to cocaine addiction and/or has been treated for cocaine addiction.

Paragraph 14. The method of any one of the preceding Paragraphs, wherein the therapeutically effective amount is effective to alleviate volitional cocaine-taking behavior.

Paragraph 15. A method, comprising administering to a subject a composition comprising an effective amount of a hydrophobic 5-hydroxytryptamine receptor 1D (5-HTR1D)-specific agonist that crosses the blood-brain barrier in the subject, wherein the subject exhibits volitional cocaine-taking behavior, and the amount is effective to reduce the volitional cocaine-taking behavior.

Paragraph 16. The method of Paragraph 15, wherein the 5-HTR1D-specific agonist binds specifically to 5-HTR1D with a Ki of less than 1000 nM, less than 100 nM, less than 10 nM, or less than 1 nM, optionally about 0.9 nM.

Paragraph 17. The method of Paragraph 16, wherein the 5-HTR1D-specific agonist comprises Compound I:

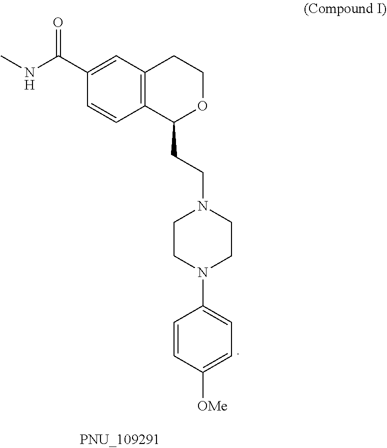 [SMILES: CNC(=O)c1ccc2c(c1)CCO[C@H]2CCN1CCN(c2ccc(OC)cc2)CC1]

Paragraph 18. The method of any one of Paragraphs 15-17, wherein the therapeutically effective amount is about 1 mg/kg to about 100 mg/kg.

Paragraph 19. The method of any one of Paragraphs 15-18, wherein the therapeutically effective amount is formulated for systemic administration or oral administration.

Paragraph 20. The method of any one of Paragraphs 15-19, wherein the composition further comprises a pharmaceutically acceptable excipient.

Paragraph 21. The method of Paragraph 20, wherein the excipient is a polyethoxylated castor oil, optionally Kolliphor®, preferably Kolliphor® HS-15.

Paragraph 22. The method of Paragraph 21, wherein the composition further comprises N-methyl-2-pyrrolidone (NMP).

Paragraph 23. The method of Paragraph 21 or 22, wherein the composition further comprises citric acid.

Paragraph 24. The method of any one of Paragraphs 15-23, wherein the excipient is equimolar to the 5-HTR1D-specific agonist.

Paragraph 25. The method of any one of Paragraphs 15-24, wherein the subject is a human.

Paragraph 26. The method of any one of Paragraphs 15-25, wherein the subject is a non-human preclinical animal model, optionally a rodent, preferably a mouse.

Paragraph 27. The method of any one of Paragraphs 15-25, wherein the subject is genetically predisposed to cocaine addiction and/or has been treated for cocaine addiction.

Paragraph 28. A composition comprising a hydrophobic 5-hydroxytryptamine receptor 1D (5-HTR1D)-specific agonist and a polyethoxylated castor oil.

Paragraph 29. The composition of Paragraph 28, wherein the polyethoxylated castor oil is Kolliphor®, optionally Kolliphor® HS-15. 520 Paragraph 30. The composition of Paragraph 29, wherein the composition further comprises N-methyl-2-pyrrolidone (NMP).

Paragraph 31. The composition of Paragraph 29 or 30, wherein the composition further comprises citric acid.

Paragraph 32. The composition of any one of Paragraphs 28, wherein the excipient is equimolar to the 5-HTR1D-specific agonist.

Paragraph 33. A method of treating cocaine addiction in a subject in need thereof, comprising administering to the subject a composition comprising a hydrophobic 5-hydroxytryptamine receptor 1D (5-HTR1D)-specific agonist, wherein a therapeutically effective amount of the 5-HTR1D-specific agonist crosses the blood-brain barrier in the subject, and the 5-HTR1D-specific agonist binds specifically to 5-HTR1D with a Ki of less than 5 nM.

Paragraph 34. The method of Paragraph 33, wherein the 5-HTR1D-specific agonist binds specifically to 5-HTR1D with a Ki of less than 1 nM.

Paragraph 35. The method of Paragraph 33, wherein the 5-HTR1D-specific agonist binds specifically to 5-HTR1D with a Ki of about 0.8 nM to about 1 nM.

Paragraph 36. The method of Paragraph 35, wherein the 5-HTR1D-specific agonist binds specifically to 5-HTR1D with a Ki of about 0.9 nM.

Paragraph 34. The method of any one of the preceding Paragraphs, wherein the 5-HTR1D-specific agonist does not bind to 5-HTR1B.

Paragraph 35. The method of any one of the preceding Paragraphs, wherein the 5-HTR1D-specific agonist binds to 5-HTR1B with a Ki of greater than 5000 nM.

Paragraph 36. The method of any one of the preceding Paragraphs, wherein the 5-HTR1D-specific agonist binds to 5-HTR1B with a Ki of greater than 5000 nM and less than 10,000 nM.

### EXAMPLES

The present disclosure is further illustrated by the following Examples. These Examples are provided to aid in the understanding of the disclosure and should not be construed as limitations thereof.

### Example 1: PNU-109291 Crosses the Blood-Brain Barrier when Injected Intravenously

An assay to test for blood-brain barrier penetration was developed based on previous work identifying that 5-HTR1D agonists directly injected into lateral ventricles of guinea pigs caused hypothermia (Skingle, Higgins, and Feniuk, 1994). Thus, if mice developed hypothermia following administration of a drug, the drug was determined to cross the blood-brain barrier. Said assay was used to test two specific 5-HTR1D agonists: PNU-109291 and zolmitriptan. PNU-109291 was tested as a candidate drug, while zolmitriptan was tested as a control known to bypass the blood-brain barrier. Both drugs were formulated using 5% Kolliphor® HS-15 and 5% NMP in saline as a vehicle, as using dimethyl sulfoxide (DMSO), a standard solvent, caused hypothermia by itself. In contrast, the vehicle composition containing 5% Kolliphor® HS-15 and 5% NMP did not cause hypothermia in mice when injected intravenously (FIG. 1, yellow line).

Mice were injected intravenously with PNU-109291 and zolmitriptan at the following doses: 0.1, 0.32, 1.0, 3.2, and 10 mg/kg mouse. The temperature of mice was then monitored for 120 minutes following injection. It was observed that 10 mg/kg doses of both PNU-109291 and zolmitriptan induced central hypothermia in C57BL/6NJ mice (FIG. 1), indicating that both drugs can bypass the blood-brain barrier.

### Example 2: PNU-109291 is Effective as a Blood-Brain Barrier-Permeable Pharmacotherapy for Volitional Aspects of Cocaine Addiction

The efficacy of PNU-109291 treatment in mice addicted to cocaine was tested. Mice first acquired cocaine IVSA at 0.75 mg/kg cocaine per dose at a fixed-ratio-1 reinforcement schedule, then a fixed-ratio-5 schedule until they stabilized their drug-taking behavior. The animals were then injected with descending doses of PNU-109291 prior to IVSA sessions. Doses of PNU-109291 were separated by vehicle injection, and vehicle-first versus agonist-first order was randomized and counterbalanced. FIG. 2A shows the significant dose effect of PNU-109291 on the total infusions of cocaine self-administered by the mice when treated with a dose of 10 mg/kg. FIG. 2B shows that the mice exhibited no differences in inactive lever presses, indicating that the phenotype was specific to volitional cocaine-taking.

What we claim is:

1. A method of treating cocaine addiction in a subject in need thereof, comprising administering to the subject a composition comprising a therapeutically effective amount of Compound I:

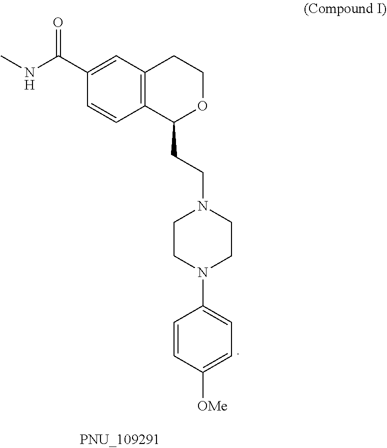 [SMILES: CNC(=O)c1ccc2c(c1)CCO[C@H]2CCN1CCN(c2ccc(OC)cc2)CC1]

2. The method of claim 1, wherein the therapeutically effective amount is about 1 mg/kg to about 100 mg/kg.

3. The method of claim 1, wherein the therapeutically effective amount is formulated for systemic administration or oral administration.

4. The method of claim 1, wherein the composition further comprises a pharmaceutically acceptable excipient.

5. The method of claim 4, wherein the excipient is a polyethoxylated castor oil.

6. The method of claim 4, wherein the excipient comprises a pH-independent non-ionic surfactant.

7. The method of claim 6, wherein the pH-independent non-ionic surfactant comprises Compound II:

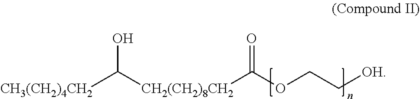

8. The method of claim 5, wherein the composition further comprises N-methyl-2-pyrrolidone (NMP), citric acid, or a combination thereof.

9. The method of claim 1, wherein the subject is a human.

10. The method of claim 1, wherein the subject is a non-human preclinical animal model, optionally a rodent, preferably a mouse.

11. The method of claim 1, wherein the subject is genetically predisposed to cocaine addiction and/or has been treated for cocaine addiction.

12. The method of claim 1, wherein the therapeutically effective amount is effective to alleviate volitional cocaine-taking behavior.

13. A composition comprising Compound I and a pH-independent non-ionic surfactant.

14. The composition of claim 13, wherein the pH-independent non-ionic surfactant comprises Compound II:

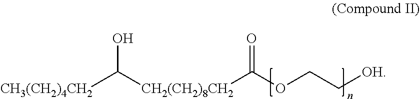

15. The composition of claim 13, wherein the composition further comprises N-methyl-2-pyrrolidone (NMP), citric acid, or a combination thereof.
